(19) United States
(12) Patent Application Publication
(10) Pub. No.: US 2025/0208118 A1
(43) Pub. Date: Jun. 26, 2025

(54) METHOD OF IDENTIFYING ORIGIN OF NON-HUMAN PRIMATE

(71) Applicant: VRL Holdings LLC, SAN ANTONIO, TX (US)

(72) Inventors: Javier Mota Sanchez, San Antonio, TX (US); Andreu Garcia Vilanova, San Antonio, TX (US); Ron Berger, San Antonio, TX (US); Dennis Pitts, San Antonio, TX (US)

(21) Appl. No.: 18/980,431

(22) Filed: Dec. 13, 2024

Related U.S. Application Data
(60) Provisional application No. 63/614,208, filed on Dec. 22, 2023.

(51) Int. Cl. G01N 33/53

(52) U.S. Cl. CPC G01N 33/53

(57) ABSTRACT

A method including collecting a sample from a non-human primate, performing a test on the sample, creating a test immune profile, providing a statistical model from control immune profiles of a known origin, comparing the test immune profile to the statistical model, and determining whether the non-human primate is of the known origin. A method including collecting samples from a plurality of non-human primates of known origin, identifying a plurality of markers in the samples, and creating a statistical model from the plurality of markers.

### CROSS REFERENCE TO RELATED APPLICATIONS

The present application claims priority to U.S. Provisional Application No. 63/614,208, filed Dec. 22, 2023, the entirety of which is incorporated herein by reference.

### FIELD OF THE INVENTION

The present method is directed toward methods of identifying origins of non-human primates.

### BACKGROUND OF THE INVENTION

The present method is directed towards methods of identifying origins of non-human primates. Non-human primates are often used in scientific and medical research. Non-human primates are valuable in medical and scientific research in part due to their proximity to human physiology, pathology, biochemistry, immune response, genetic evolution, and other traits. When selecting non-human primates for scientific and medical research, the origins thereof can impact the data produced from the research, the financial value of the non-human primates, the legality of use of the non-human primates, and other factors.

### SUMMARY OF THE INVENTION

In embodiments, a method comprises collecting a sample from a non-human primate; performing a test on the sample, wherein the test identifies a plurality of markers; creating a test immune profile of the plurality of markers present in the non-human primate; providing a database comprising control immune profiles of a known origin; creating a statistical model from the database comprising the control immune profiles; comparing the test immune profile to the statistical model; and determining whether the non-human primate comprises the known origin, wherein the non-human primate is identified as comprising the known origin if the test immune profile substantially matches the statistical model and the non-human primate is identified as comprising a second known origin if the test immune profile does not substantially match the statistical model.

In embodiments, a method comprises collecting a sample from a non-human primate; performing a test on the sample, wherein the test identifies a plurality of markers; creating a test immune profile of the plurality of markers identified in the test; providing at least one statistical model comprising control immune profiles of a known origin; and comparing the test immune profile to the at least one statistical model; wherein, if the test immune profile substantially matches the at least one statistical model, then the origin of the non-human primate is identified as comprising the known origin; and wherein, if the test immune profile does not substantially match the at least one statistical model, then the origin of the non-human primate is identified as not comprising the known origin.

In embodiments, a method comprises providing a plurality of known wild-caught or captive-bred non-human primates; collecting samples from the plurality of known wild-caught or captive-bred non-human primates; identifying a plurality of markers in the samples; and creating a statistical model from the plurality of markers identified, wherein the statistical model comprises data characteristic of wild-caught or captive-bred non-human primates.

In embodiments, the sample comprises a blood sample. In embodiments, the test is performed on serum separated from the blood sample.

In embodiments, the non-human primates of known origin comprise wild-caught non-human primates. In embodiments, the non-human primates of known origin comprise captive-bred non-human primates.

In embodiments, the method further comprises providing a second database comprising control immune profiles of the second known origin; and creating a second statistical model from the database comprising the control immune profiles of the second known origin.

In embodiments, the at least one statistical model comprises a first statistical model and a second statistical model. In embodiments, the method further comprises providing the second statistical model comprising second control immune profiles of a second known origin; and comparing the test immune profile to the second statistical model if the test immune profile does not substantially match the first statistical model; wherein, if the test immune profile substantially matches the second statistical model, then the origin of the non-human primate is identified as comprising the second known origin.

In embodiments, the method further comprises providing a second plurality of known wild-caught or captive-bred non-human primates; collecting samples from the second plurality of known wild-caught or captive-bred non-human primates; identifying a second plurality of markers in the samples from the second plurality of known wild-caught or captive bred non-human primates; and creating a second statistical model from the second plurality of markers identified, wherein the statistical model comprises data characteristic of wild-caught or captive-bred non-human primates. In embodiments, at least one plurality of known wild-caught non-human primates are provided and at least one plurality of known captive-bred non-human primates is provided.

In embodiments, the statistical model comprises a heatmap.

In embodiments, when the non-human primate is identified as being of the known origin, the non-human primate is verified as complying with at least one legal requirement and is transported to a facility.

### BRIEF DESCRIPTION OF THE DRAWINGS

FIG. 1 is a heatmap of control immune profiles.

### DETAILED DESCRIPTION

In the following detailed description of embodiments, reference is made to the accompanying drawings, which form a part hereof and in which are shown, by way of illustration, specific embodiments in which the invention may be practiced. Specific details disclosed herein are in every case a non-limiting embodiment representing concrete ways in which the concepts of the invention may be practiced. This serves to teach one skilled in the art to employ the present invention in virtually any appropriately detailed system, structure or manner consistent with those concepts. It will be seen that various changes and alternatives to the specific described embodiments and the details of those embodiments may be made within the scope of the invention. Because many varying and different embodiments may be made within the scope of the inventive concepts herein described and in the specific embodiments herein detailed without departing from the scope of the present invention, it is to be understood that the details herein are to be interpreted as illustrative and not as limiting.

The various directions such as “upper,” “lower,” “bottom,” “top,” “back,” “front,” “perpendicular”, “vertical”, “horizontal,” “length” and width” and so forth used in the detailed description of embodiments are made only for easier explanation in conjunction with the drawings to express the concepts of the invention. The elements in embodiments may be oriented differently while performing the same function and accomplishing the same result as obtained with the embodiments herein detailed, and such terminologies are not to be understood as limiting the concepts which the embodiments exemplify.

As used herein, the use of the word “a” or “an” when used in conjunction with the term “including” (or the synonymous “may include” or “including”) in the claims and/or the specification may mean “one,” but it is also consistent with the meaning of “one or more,” “at least one,” and “one or more than one.” In addition, as used herein, the phrase “connected to” means joined to or placed into communication with, either directly or through intermediate components.

Embodiments of the present method include providing a plurality of known wild-caught or captive-bred non-human primates, collecting samples from the plurality of known wild-caught or captive-bred non-human primates, identifying a plurality of markers in the samples, and creating a statistical model from the plurality of markers identified.

Embodiments of the present method include collecting a sample from a non-human primate, performing a test on the sample, creating a test immune profile, providing a database, creating a statistical model from the database, comparing the test immune profile to the statistical model, and identifying an origin of the non-human primate.

Non-human primates may be of known or unknown origin. Origin may include a non-human primate's bred status, geographical region, captive breeder, or vendor.

The method may include collecting a sample from a non-human primate. The sample may include at least one of a blood sample, a saliva sample, a urine sample, a stool sample, and any suitable biological sample. When the sample is a blood sample, serum may be separated from a remaining portion of the blood sample. The sample may be processed in any suitable manner.

The method may include performing a test on the sample collected from the non-human primate. The test may identify a plurality of markers. Markers may include antigens, antibodies, bacteria, viruses, parasites, chemicals, proteins, peptides, immunoglobulins, biomarkers, DNA, RNA, probes, metabolites, and toxins. The test may be performed on at least one of the blood sample, the serum, the saliva sample, the urine sample, the stool sample, and the suitable biological sample. The test may include an antigen test.

The method may include creating a test immune profile. The test immune profile may include the plurality of markers identified in the test. The test immune profile may include a test antigen profile.

The method may include providing a plurality of non-human primates of known origin. The method may include providing a plurality of non-human primates of a plurality of known origins, including at least a first known origin and a second known origin. The known origin or plurality of known origins may include wild-caught status, captive-bred status, a specific wild-caught geographic region, a specific captive breeder, or a specific vendor. The plurality of known origins may include different origins or overlapping origins.

The method may include collecting samples from the plurality of non-human primates of known origin. The samples may include at least one of a blood sample, a saliva sample, a urine sample, a stool sample, and any suitable biological sample. When the samples are blood samples, serum may be separated from a remaining portion of the blood samples. The samples may be processed in any suitable manner.

The method may include identifying a plurality of markers in the samples taken from the plurality of non-human primates of known origin. The method may include identifying a plurality of markers of non-human primates of a plurality of known origins, including at least the first known origin and the second known origin. The method may include identifying a first plurality of markers in the samples taken from the non-human primates of the first known origin. The method may include identifying a second plurality of markers in the samples taken from the non-human primates of the second known origin. second plurality of markers in the samples. Markers may include antigens, antibodies, bacteria, viruses, parasites, chemicals, proteins, peptides, immunoglobulins, biomarkers, DNA, RNA, probes, metabolites, and toxins. The plurality of markers may be identified by performing a test. The test may be performed on at least one of the blood samples, the serum, the saliva samples, the urine samples, the stool samples, and the suitable biological samples. The plurality of markers may include markers known to be characteristic of the known origin. The plurality of markers may include markers determined to be characteristic of the known origin by performance of the method. The plurality of markers may include markers that are known to not be characteristic of at least one additional known origin. The plurality of markers may include markers that are determined to not be characteristic of at least one additional known origin by performance of the method.

The method may include providing a database. The database may include control immune profiles of a known origin. The known origin may include wild-caught status, captive-bred status, a specific wild-caught geographic region, a specific captive breeder, or a specific vendor. The method may include providing a plurality of databases, including at least a first and a second database. The first database may include control immune profiles of a first known origin. The second database may include control immune profiles of a second known origin. The first known origin and the second known origin may include wild-caught status, captive-bred status, a specific wild-caught geographic region, a specific captive breeder, or a specific vendor. The first known origin and the second known origin may include different or overlapping origins. The plurality of known origins may include at least one wild-caught origin and at least one captive-bred origin.

The method may include creating a statistical model. The statistical model may include control immune profiles of a known origin. The statistical model may include a statistical model based on data contained in the database of control immune profiles. The statistical model may include data characteristic of the known origin. The method may include creating a plurality of statistical models, including at least a first statistical model and a second statistical model. The first statistical model may be created from the first database. The first statistical model may include control immune profiles of a first known origin. The first statistical model may include data characteristic of the first known origin. The second statistical model may be created from the second database. The second statistical model may include control immune profiles of a second known origin. The second statistical model may include data characteristic of the second known origin. The statistical model may include a heatmap, cluster model, dot distribution map, scatter plot, density model, decision tree, deep learning network, generalized linear model, gradient boosting machine, Naïve Bayes, XGBoost tree, any suitable machine learning algorithm, any suitable visualization model, or any suitable statistical model. The plurality of statistical models may include heatmaps, cluster models, dot distribution maps, scatter plots, density models, decision trees, deep learning networks, generalized linear models, gradient boosting machines, Naïve Bayes, XGBoost trees, any suitable machine learning algorithms, any suitable visualization models, or any suitable statistical models.

The method may include comparing the test immune profile to the statistical model. The test immune profile may be compared to the statistical model in any suitable manner, including by running data from the test immune profile through an algorithm and creating a model of the test immune profile of the same type as the statistical model of the control immune profiles of the known origin.

The method may include determining whether the non-human primate originates from the known origin, does not originate from the known origin, or originates from an additional known origin. The non-human primate may be of an unknown, uncertain, or unconfirmed origin. By comparing the test immune profile of the plurality of markers present in the non-human primate, the non-human primate may be identified as being of the known origin, not of the known origin, or being of an additional known origin. The non-human primate may be identified as being of the known origin if the test immune profile substantially matches the statistical model created from the database of the control immune profiles. The non-human primate may be identified as not being of the known origin if the test immune profile does not substantially match the statistical model created from the database of the control immune profiles. The non-human primate may be identified as being of a second known origin if the test immune profile does not substantially match the statistical model created from the database of the control immune profiles. The non-human primate may be identified as being of the second known origin if the test immune profile substantially matches a second statistical model created from a second database of a second set of control immune profiles.

The method may include verifying that the non-human primate complies with at least one legal requirement. The method may include verifying that the non-human primate is of a known origin, as required by law. The at least one legal requirement may include any past, present, or future legal requirement or combination thereof including, but not limited to, at least one state law, at least one federal law, at least one non-United States law, and any combination thereof. The at least one legal requirement may be substantially related to the requirements set forth in 42 CFR § 71.53.

The method may include transporting the non-human primate to a facility. The facility may include any suitable facility including, but not limited to, a breeding colony, a broker, an educational facility, an exhibition, a laboratory, a Centers for Disease Control and Prevention (“CDC”) quarantine, or any other facility that has a permitted purpose for use and/or possession of a non-human primate. The facility may be located anywhere including, but not limited to, any state, territory, or other region in the United States. The facility may otherwise be located outside of the United States and its territories.

### Example #1

In a first specific embodiment, blood samples were taken from over 200 non-human primates of different geographic origins and bred statuses, namely, captive-bred or wild-caught. These blood samples were used to identify markers correlated with the different geographic origin and bred status. Membranes of appropriate size and composition to perform an assay of the samples were provided. Markers were dotted on the nitrocellulose membranes and allowed to dry. The nitrocellulose membranes were blocked with a blocking solution for an hour at room temperature with slow agitation. The membranes were washed with a blocking solution at least three times. The membranes were incubated for one hour at room temperature with the samples diluted in a sample diluent. The membranes were washed with wash solutions at least three times. The membranes were incubated for at least one hour with detection antibodies at room temperature with slow agitation. The membranes were washed with wash solutions at least three times. The membranes were washed with a second wash solution to substantially remove contaminants. The membranes were incubated at room temperature with detection reagents until substantially ready and washed with wash solution once more before imaging and storage.

The membranes were scanned, resulting in membrane scans. The membrane scans were processed, resulting in images. The images were trimmed, processed, and formatted. The images were processed to identify positions and normalize the image data to background data. The images included immunoblot spots. The immunoblot spots were analyzed for normalized intensity and area. The area and intensity of the immunoblot spots was calculated to quantify sample reactivity against the identified markers correlated with the different geographic origins and bred statuses. A data analysis pipeline classified the samples as being of certain geographic origin and/or bred status based on their reactivity to the markers correlated with the different geographic origins and bred statuses.

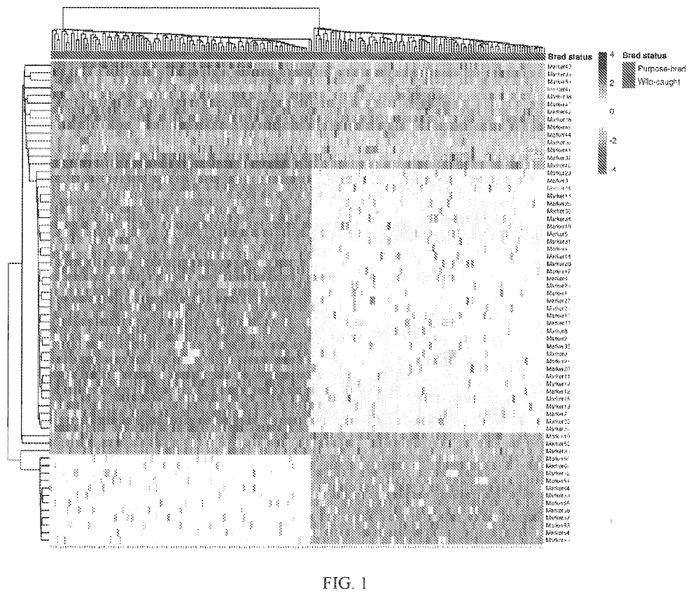

The classified samples were each organized in a grid. Digitalized images including the immunoblot spots of the grid were generated and used to calculate the intensity and size of the immunoblot spot. The grid of each sample was used to define a heatmap. The heatmap included clusters of grids of various samples which shared a the same or a similar marker profile based on the intensity and size of their respective immunoblot spots. A representative heatmap is shown in FIG. 1. The heatmap identified at least two groups of non-human primates of different geographic origin and/or bred status, two corresponding to a first vendor and one corresponding to a second vendor.

### Example #2

In a second specific embodiment, serum and saliva samples from 50 non-human primates will be received in a laboratory. A microtiter plate with a matrix will be provided. The matrix will have proteins, peptides, and nucleic acids printed and blocked to avoid non-specific interactions. The samples will be diluted using sample diluents and introduced into specific wells and allowed to bind for at least an hour. The samples will be washed using an automatic washer and incubated with fluorescently labeled anti-non-human primate antibodies and probes for at least an hour. The microtiter plate will be washed. The fluorescent signal will be stabilized using a signal stabilizer media.

After the stabilizer media solidifies, the plates will be imaged using an automated microscope. Reference analytes will be used to generate a virtual matrix location. each positive signal will be correlated with a position in the matrix and a marker assigned to the position. Median Fluorescent Intensity (MFI) will be calculated for each marker and detection type. An immune profile will be built for each sample.

The immune profile of each sample will be classified as a purpose-bred or wild-caught non-human primate using a machine learning algorithm. The classification of each sample will have a confidence interval of the non-human primate being purpose-bred or wild-caught. A final report will be generated. The final report will have information relating to which non-human primates are certified purpose-bred, including the associated confidence interval. The final report will be provided to a customer. The remainder of the samples will be stored for potential retesting.

### Example #3

In a third specific embodiment, a customer will receive a shipment of 20 non-human primates from a vendor. Screening kits and a lateral flow reader will be provided to the customer for the non-human primates. The screening kits will have 5×5 matrices of 25 markers printed on lateral-flow membranes and a QR barcoded support material. The customer will take blood samples from the non-human primates. The customer will separate serum samples from the blood samples. The serum samples will be diluted with a diluent. The customer will drop the diluted serum samples on the membrane. Reagents will be present in the lateral-flow membranes and will be activated upon application of the serum samples to the lateral-flow membranes. The diluted serum samples will bind and react with the markers printed on the membranes. Within 25 minutes, results from the membranes will be ready to analyze.

The customer will use the lateral flow reader to capture digital images of the matrices. The digital images associated with each sample will be sent to a vendor for classification as purpose-bred or wild-caught. The vendor will classify the samples using a classification algorithm. A report of the classification of the samples will be sent to the customer.

What is claimed is:

1. A method comprising:
collecting a sample from a non-human primate;
performing a test on the sample, wherein the test identifies a plurality of markers;
creating a test immune profile of the plurality of markers present in the non-human primate;
providing a database comprising control immune profiles of a known origin;
creating a statistical model from the database comprising the control immune profiles;
comparing the test immune profile to the statistical model; and
determining whether the non-human primate comprises the known origin, wherein the non-human primate is identified as comprising the known origin if the test immune profile substantially matches the statistical model and the non-human primate is identified as comprising a second known origin if the test immune profile does not substantially match the statistical model.

2. The method of claim 1, wherein the sample comprises a blood sample.

3. The method of claim 2, wherein the test is performed on serum separated from the blood sample.

4. The method of claim 1, wherein the non-human primates of known origin comprise wild-caught non-human primates.

5. The method of claim 1, wherein the non-human primates of known origin comprise captive-bred non-human primates.

6. The method of claim 1, further comprising:
providing a second database comprising control immune profiles of the second known origin; and
creating a second statistical model from the database comprising the control immune profiles of the second known origin.

7. The method of claim 1 wherein the statistical model comprises a heatmap.

8. A method comprising:
collecting a sample from a non-human primate;
performing a test on the sample, wherein the test identifies a plurality of markers;
creating a test immune profile of the plurality of markers identified in the test;
providing at least one statistical model comprising control immune profiles of a known origin; and
comparing the test immune profile to the at least one statistical model;
wherein, if the test immune profile substantially matches the at least one statistical model, then the origin of the non-human primate is identified as comprising the known origin;
wherein, if the test immune profile does not substantially match the at least one statistical model, then the origin of the non-human primate is identified as not comprising the known origin; and
wherein, when the non-human primate is identified as comprising the known origin, the non-human primate is verified as complying with at least one legal requirement and is transported to a facility.

9. The method of claim 8, wherein the sample comprises a blood sample.

10. The method of claim 9, wherein the test is performed on serum separated from the blood sample.

11. The method of claim 8, wherein the non-human primates of known origin comprise wild-caught non-human primates.

12. The method of claim 8, wherein the non-human primates of known origin comprise captive-bred non-human primates.

13. The method of claim 8, wherein the statistical model comprises a heatmap.

14. The method of claim 8, wherein the at least one statistical model comprises a first statistical model and a second statistical model, wherein the method further comprises:
providing the second statistical model comprising second control immune profiles of a second known origin; and
comparing the test immune profile to the second statistical model if the test immune profile does not substantially match the first statistical model;
wherein, if the test immune profile substantially matches the second statistical model, then the origin of the non-human primate is identified as comprising the second known origin.

15. A method comprising:
providing a plurality of known wild-caught or captive-bred non-human primates;
collecting samples from the plurality of known wild-caught or captive-bred non-human primates;
identifying a plurality of markers in the samples; and
creating a statistical model from the plurality of markers identified, wherein the statistical model comprises data characteristic of wild-caught or captive-bred non-human primates.

16. The method of claim 15, wherein the samples comprise blood samples.

17. The method of claim 16, wherein the plurality of markers are identified from serum separated from the blood samples.

18. The method of claim 15, further comprising:
providing a second plurality of known wild-caught or captive-bred non-human primates;
collecting samples from the second plurality of known wild-caught or captive-bred non-human primates;
identifying a second plurality of markers in the samples from the second plurality of known wild-caught or captive bred non-human primates; and
creating a second statistical model from the second plurality of markers identified, wherein the statistical model comprises data characteristic of wild-caught or captive-bred non-human primates.

19. The method of claim 18, wherein at least one plurality of known wild-caught non-human primates are provided and at least one plurality of known captive-bred non-human primates is provided.

20. The method of claim 15, wherein the statistical model comprises a heatmap.

21. The method of claim 1, wherein, when the non-human primate is identified as comprising the known origin, the method further comprises:
verifying that the non-human primate complies with legal requirements; and
transporting the non-human primate to a facility.
